(19) United States
(12) Patent Application Publication
(10) Pub. No.: US 2005/0004430 A1
(43) Pub. Date: Jan. 6, 2005

(54) Endoscopic balloon insertion device for treatment of obesity and insertion technique of the same

(76) Inventors: Jung Hwan Lee, Seoul (KR); Kook Hwan Lee, Seoul (KR)

Correspondence Address: LADAS & PARRY LLP, 224 SOUTH MICHIGAN AVENUE, SUITE 1200, CHICAGO, IL, 60604, US

(21) Appl. No.: 10/867,511

(22) Filed: Jun. 14, 2004

(30) Foreign Application Priority Data
Jun. 26, 2003 (KR) 10-2003-0042270

(51) Int. Cl. A61B 1/00

(52) U.S. Cl. 600/116; 606/151; 606/191

(57) ABSTRACT

The present invention relates to a treatment of obesity, and in particular to a therapeutic method belonging to a surgical field for obesity wherein it is possible to reduce a calorie intake amount and to lose weight using a gastroscope used for endoscopic examination of the stomach in such a manner that a certain balloon is inserted in the interior of stomach of fat person for thereby decreasing the entire volume of stomach, so that a user feels a earlier fullness during eating food thus decreasing the amount of food intake.

### BACKGROUND OF THE INVENTION

1. Field of the Invention

The present invention relates to a treatment of obesity, and in particular to a therapeutic method belonging to a surgical field for obesity wherein it is possible to reduce a calorie intake amount and to lose weight using a gastroscope for endoscopic examination of the stomach in such a manner that a certain balloon is inserted in the interior of stomach of fat person for thereby decreasing the entire volume of stomach, so that a user feels earlier fullness during eating food thus decreasing the amount of food intake.

2. Description of the Background Art

Generally, the treatment of obesity is classified in the following three categories.

First, it is an exercise and diet method that is well recognized to people. If the intake amount of calorie is more than the consumption amount, over calorie is accumulated as fat in the body. In this case, it is needed to burn more calories with exercise based on the above fatness principle. However, this method needs endless efforts and patience, and it is impossible to achieve a desired effect for short time period. In addition, this method is not actually adapted to a high fat person, e.g. severe obese person, who has problems for exercise.

The second therapy is directed to a medical therapy. There are two kinds of medicines of Xenical and Reductil as a fatness therapeutic agent authorized by the US FDA. Here, Xenical is an agent capable of suppressing intestinal absorption of fat. When it is taken as medicine together with food, the absorption of fat into body is suppressed for thereby losing weight. However, it should be always taken after eating food, and fat feces is outputted for thereby causing inconvenience.

In addition, when taking of it is stopped, the effect of the same disappears. A certain side effect of the same is not verified. In addition, Reductil is an agent capable of inducing weight reduction by decreasing appetite of patient in such a manner that the appetite center of central nervous system is suppressed. It is taken one time per day, so it is convenient. There is not any side effect of fat feces like Xenical. When taking of medicine is stopped, the effect is maintained for a certain time period. However, Reductil is a kind of psychotropic drug. Therefore, there may be another side effect in connection with the psychotropic drug. When it is taken for a long time period, chronic anorexia may occur. A certain abnormal problem may occur in body.

The third therapeutic method is directed to a bariatric surgery that is limitedly performed with respect to a severe obese patient in the US. This method is achieved based on two principles. Namely, there is one principle in which a part of stomach is resected, and the entire volume of stomach is decreased, and an earlier fullness and eating suppression are provoked for thereby losing the weight. There is the other principle in which a part of small bowel is resected, and an absorption problem, e.g. malabsorption, occurs for thereby losing the weight. However, the above operative methods have potential dangers due to an operation. In addition, when it is performed once, it is impossible to restore to the original state, and if the therapy fails, the operation should be performed repeatedly. The above operations are very limitedly performed in the case that the therapy is failed by two methods.

### SUMMARY OF THE INVENTION

Accordingly, it is a first object of the present invention to overcome the problems encountered in the conventional treatment of obesity.

It is a second object of the present invention to provided a method based on a surgical method in which a certain balloon is inserted into a stomach using an endoscope (Q240 or two channel endoscope manufactured by Olympus company) that has been generally used in the hospitals without open surgery thereby decreasing the volume of stomach. For reference, FIG. 10-A, 10-B, 10-C shows a principle and structure of endoscope generally used for a diagnosis and therapy in the hospitals.

It is a third object of the present invention to achieve a stable operation using endoscope (without side effect in operation wherein it is possible to finish the operation within 30 minutes).

It is a fourth object of the present invention to achieve a desired purpose in such a manner that general anesthesia performed during an open surgery, e.g. bariatric surgery is not needed, and only pre-medication same as a routine endoscopic examination is needed, and in some cases, a pre-medication such as a conscious sedative endoscopy is needed.

It is a fifth object of the present invention to achieve a perfect recovery in such a manner that a balloon inserted in stomach can be retrieved when it is needed to remove the balloon using the endoscope in the case that a desired weight reduction is achieved or a therapy is failed.

It is a sixth object of the present invention to prevent any gastric obstruction because the balloon is movable in the stomach, so that the movement of food is not interfered.

It is a seventh object of the present invention to achieve a desired weight reduction with respect to people who want a weight reduction wherein high fat people are not limited like bariatric surgery.

It is an eight object of the present invention to achieve more effects in cooperation with an exercise and diet.

To achieve the above objects, in an endoscopic technique in which a balloon is inserted into a stomach for a fatness therapy, there is provided an endoscopic balloon insertion method, comprising a step in which an endoscope is inserted into a stomach, and a guide pipe with a balloon is inserted into the stomach through a biopsy channel of the endoscope; a step in which the balloon is separated from the guide pipe inserted into the stomach through the endoscope; a step in which the balloon is released in the stomach through the endoscope, and the balloon is destroyed and retrieved from the stomach out of body; and a step in which when a guide pipe having a larger diameter is used, a guide pipe is inserted into the stomach using a X-ray fluoroscope, and an endoscope is inserted along a lateral side of the same for thereby releasing the balloon in the stomach.

To achieve the above objects, in an endoscopic insertion device capable of inserting a balloon into a stomach for a fatness therapy, there is provided an endoscopic insertion apparatus, comprising a certain shaped balloon that is composed of a certain material strong to gastric acid and does not interfere passage of foods and achieve a volume decrease effect of stomach; inner and outer guide pipes in which a balloon is engaged between a front end of each of the same; a rubber band engaged to the guide pipes for elastically sealing the balloon when the balloon is released from the guide pipes; and a guide pipe having the same structure as the guide pipes and having a diameter larger than that of the guide pipes.

### BRIEF DESCRIPTION OF THE DRAWINGS

The present invention will become better understood with reference to the accompanying drawings which are given only by way of illustration and thus are not limitative of the present invention, wherein;

FIG. 1 is a cross sectional view illustrating the entire structure of a guide pipe having a balloon according to the present invention;

FIG. 2 is a view illustrating the construction that a guide pipe is inserted into a biopsy channel of an endoscope and is extended to the outside of a front end of an endoscope according to the present invention;

FIG. 3 is a view illustrating an escape of a rubber band after air is inputted into a balloon, and the balloon is engaged between inner and outer guide pipes and is expanded according to the present invention, wherein a dotted line represents the position of rubber band escaped by pushing handle of outer guide pipe, and there is shown a cross section of inner and outer guide pipes;

FIG. 4 is a cross sectional view of an end portion of a guide pipe according to the present invention wherein a balloon is bound on the inner pipe by a rubber band is popped outside when a pushing handle is pushed after air is inserted into a balloon by a syringe;

FIGS. 5 and 6 are views illustrating the cross sectional views of a front end of a guide pipe and a movement of a rubber band according to the present invention;

FIG. 7 is a view illustrating an operation that a balloon is inserted into a stomach according to the present invention wherein only separated balloon is shown therein;

FIG. 8 is a view of a method that a guide pipe is inserted into a stomach, and an endoscope is inserted in a lateral side for thereby checking an expansion of balloon wherein the above method is used when a guide pipe is not inserted into a biopsy channel because the diameter of a guide pipe is large according to the present invention;

FIG. 9 is a view of a method when a balloon is removed according to the present invention in such a manner that a balloon is destroyed by inserting a cutting needle knife which is inserted into a biopsy channel of an endoscope, and then the balloon is retrieved by a retrieval forcep according to the present invention; and

FIG. 10-A, 10-B, 10-C are view of a principle and structure of an endoscope used for a diagnosis and therapy used in the hospitals.

### DETAILED DESCRIPTION OF THE PREFERRED EMBODIMENTS

The preferred embodiments of the present invention will be described with reference to the accompanying drawings.

1. The Method of Using a Biopsy Channel of Endoscope

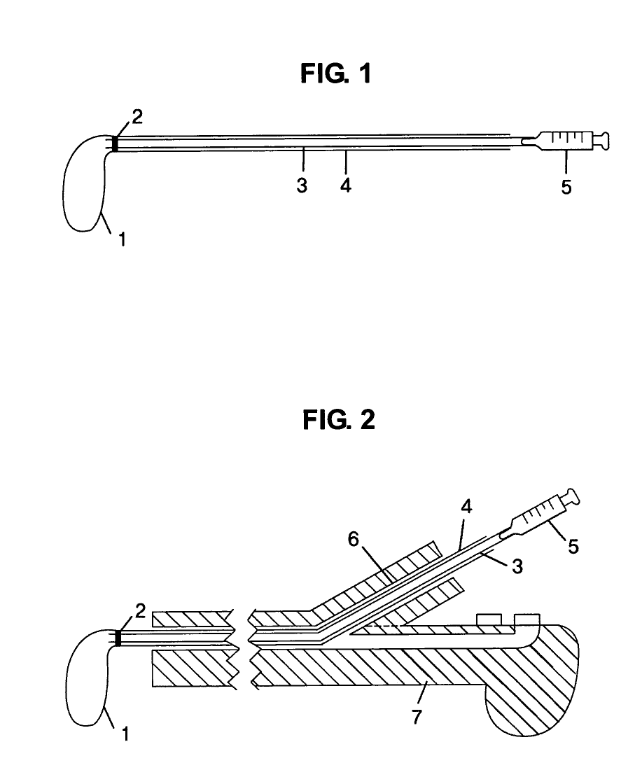

As shown in FIG. 1, a guide pipe having a diameter of 0.3 cm in maximum is inserted into a biopsy channel of an endoscope wherein a balloon 1 is engaged thereto. In the structure of a guide pipe, an inner guide pipe 3 and an outer guide pipe 4 are hollow. A balloon is attached between each front end of the same. A rubber band 2 having a high elastic force is provided in the pipe for fixing a balloon thereto. A syringe 5 is inserted into the other end portion of the inner guide pipe for injecting air thereinto for thereby expanding the balloon.

As shown in FIG. 2, a guide pipe is inserted through a biopsy channel 6 of a two-channel endoscope 7. In this operation method, an endoscope is inserted into a patient's stomach based on routine endoscopic examination, and a guide pipe with a balloon is inserted through a biopsy channel for thereby expanding a balloon.

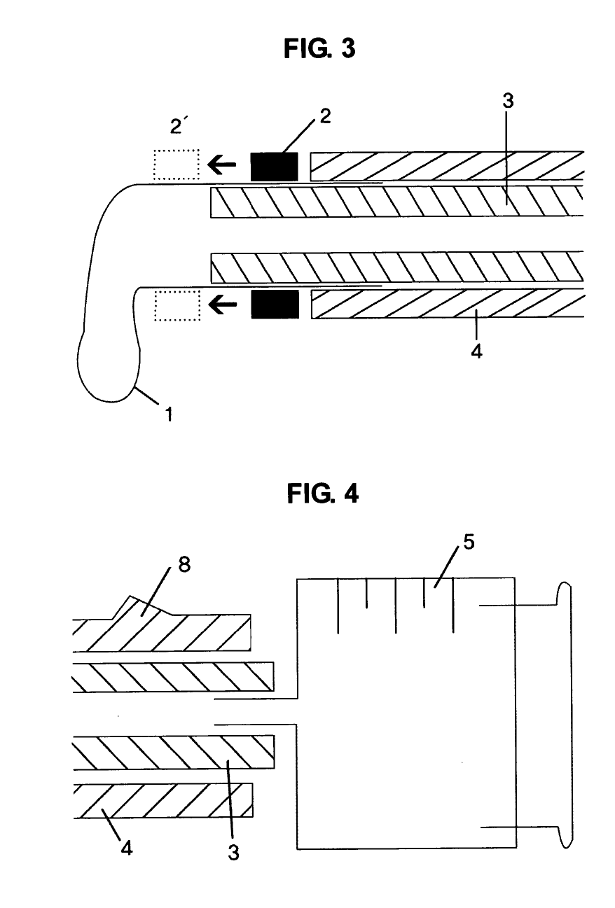

As shown in FIG. 3, a rubber band 2 with a high elastic force surrounds the inner guide pipe 3 for stably binding an opening of the balloon when the balloon 1 is expanded. The outer guide pipe 4 is positioned at a rear portion of the rubber band. As shown in FIG. 4, the syringe 5 is engaged at the other end portion of the inner guide pipe. A proper amount of air is inputted for thereby expanding the balloon. In this state, when the pushing handle 8 of the rear portion of the guide pipe 4 is pushed in the direction of the front end, the rubber band is moved in the direction of the front end of the inner guide pipe by a front end of the outer guide pipe and is escaped from the guide pipe. Therefore, the escaped rubber band seals and releases the balloon from the guide pipe for thereby inserting the balloon in the stomach in a state that the balloon is tied by the rubber band.

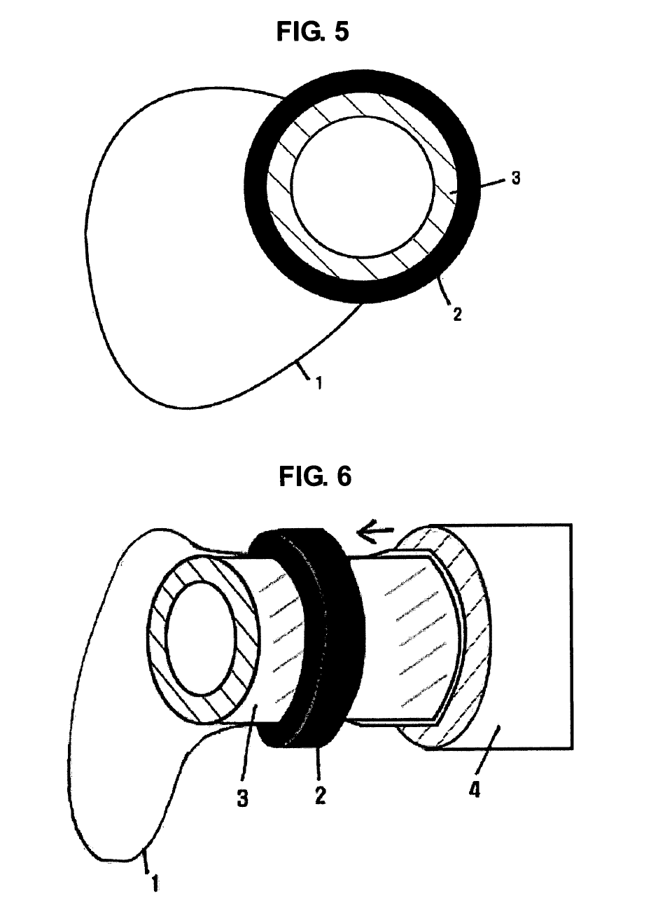

FIG. 5 is a cross sectional view of the rubber band 2 on the inner guide pipe 3. As shown in FIG. 6, when the pushing handle 8 is pushed, the rubber band is forwardly moved and escaped based on the forward movement of the outer guide pipe 4.

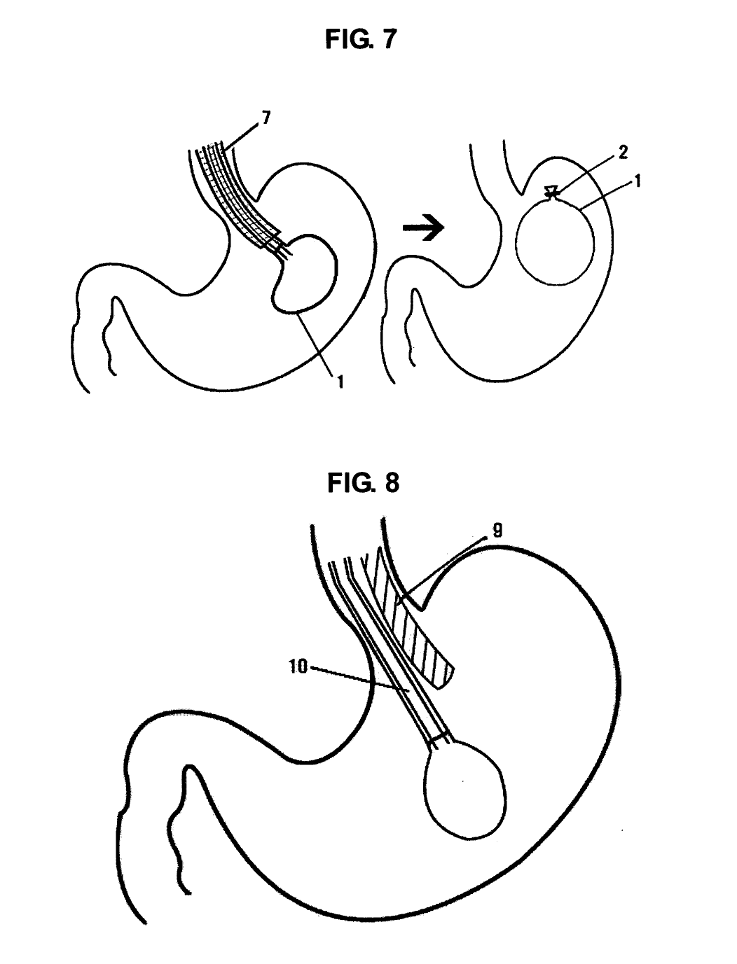

FIG. 7 is a view illustrating the guide pipe with the balloon 1 through a biopsy channel of the endoscope 7 inserted into a patient's stomach. In the above state, when the balloon is expanded, the balloon is released within the stomach. When the operation is finished, the guide pipe and the endoscope are retrieved out of body.

2) Method of Using Lateral Side of Endoscope

In the case that the guide pipe 10 having a diameter larger than the guide pipe used in the method 1 is used, since it is impossible to insert the guide pipe into the biopsy channel of the endoscope, the following method is preferably adapted.

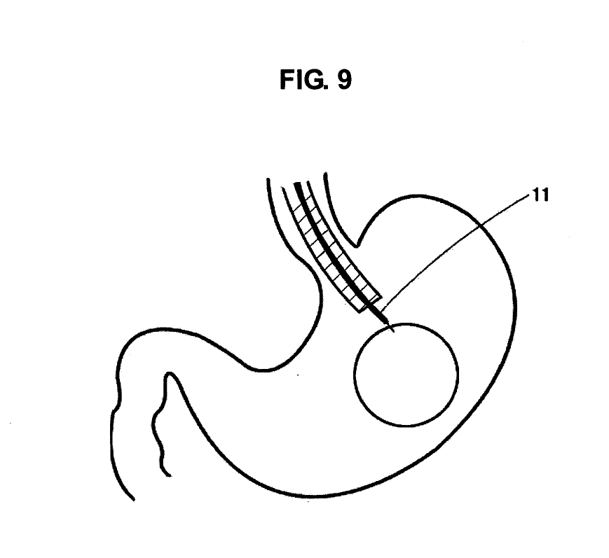

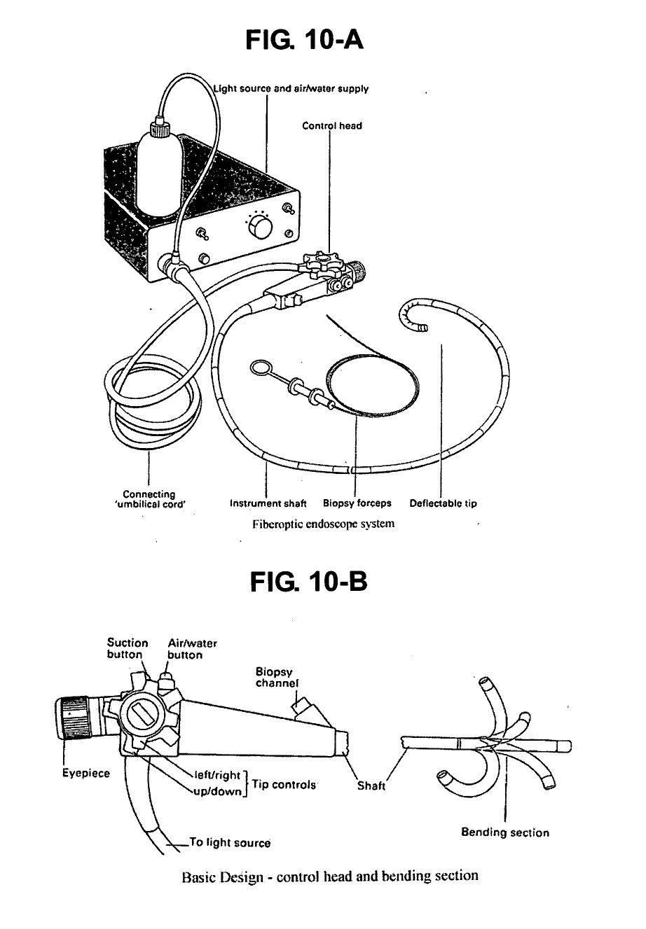

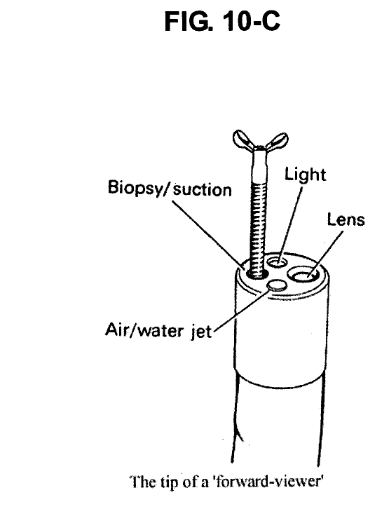

As shown in FIG. 8, the guide pipe 10 having a diameter larger than the guide pipe of the method 1 is inserted into the stomach through a patient's mouth using a X-ray fluoroscope. In addition, the Q240 endoscope 9 by Olympus company that has been generally used for the purpose of diagnosis is inserted along a lateral side of the guide pipe. The above procedure is performed for checking an expansion and release of the balloon in the stomach. The expanding and release methods of the balloon are performed in the same manner as the method 1. When the operation is finished, the guide pipe and endoscope are retrieved out of body.

3) Method of Removing Balloon

In the case that a desired therapeutic effect is obtained or the therapy is failed, the inserted balloon is easily removed by the following methods.

A cutting needle knife 11 is inserted into the biopsy channel of the endoscope using the therapeutic endoscopic method that has been generally performed in the hospitals for thereby cutting and perforating the balloon inserted in the interior of the stomach.

When the cutted balloon is perforated and destroyed and gets smaller, the retrieval forcep is inserted into the biopsy channel of the endoscope for thereby grasping and retrieving the destroyed balloon.

As described above, in the present invention, it is possible to insert a balloon into the stomach using an endoscope and a guide pipe with a balloon for thereby easily decreasing the volume of stomach. As one of the most advantageous matters, the present invention is invasive like surgery, however, this invention is capable of achieving a full recovery of stomach to an original size, and the therapeutic effect is similar with the bariatric surgery or is better than the same. As another advantage, in the case that the patient wants weight reduction, the present invention may be adapted to all of the fat person. In addition, the present invention may be implemented together with exercise and diet. In the future, the following matters should be considered. First, a certain damage should not occur in the stomach. Second, it is needed to develop a balloon made of a material strong to gastric acid. Third, what kinds of shape (tube shape, circular shape, elliptical shape, etc.) of balloon are proper, not interfering the movement of foods in stomach, for thereby effectively decreasing the volume of stomach. In addition, a certain symptom such as continuous abdominal fullness that may occur after inserting the balloon into stomach should be prevented.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. In a technique of endoscopic therapy in which a balloon is inserted into a stomach for a treatment of obesity, an endoscopic balloon insertion method, comprising:
a step in which an endoscope is inserted into a stomach, and a guide pipe with a balloon is inserted into the stomach through a biopsy channel of the endoscope;
a step in which the balloon is separated from the guide pipe inserted into the stomach through the endoscope;
a step in which the balloon is released in the stomach through the endoscope, and the balloon is destroyed and retrieved from the stomach out of body; and
a step in which when a guide pipe having a larger diameter is used, a guide pipe is inserted into the stomach using a X-ray fluoroscope, and an endoscope is inserted along a lateral side of the same for thereby releasing the balloon in the stomach.

2. In an endoscopic insertion device capable of inserting a balloon into a stomach for a treatment of obesity, an endoscopic balloon insertion apparatus, comprising:
a certain shaped balloon 1 that is composed of a certain material strong to gastric acid and does not interfere passage of foods, and achieve a volume decrease effect of stomach;
inner and outer guide pipes 3 and 4 in which a balloon is engaged between a front end of each of the same;
a rubber band 2 engaged to the guide pipes for elastically sealing the balloon when the balloon is released from the guide pipes; and
a guide pipe 10 having the same structure as the guide pipes 3 and 4 and having a diameter larger than that of the guide pipes 3 and 4.
